(19) United States
(12) Patent Application Publication
(10) Pub. No.: US 2014/0288279 A1
(43) Pub. Date: Sep. 25, 2014

(54) M-DC8+ Monocyte Depleting Agent for the Prevention or the Treatment of a Condition Associated with a Chronic Hyperactivation of the Immune System

(71) Applicants: Institut National de la Sante et de la Recherche Medicale (INSERM), Paris (FR); Centre National de la Recherche Scientifique (CNRS, Paris (FR)

(72) Inventors: Anne Hosmalin, Paris (FR); Charles-Antoine Dutertre, Cedex 18 Paris (FR)

(21) Appl. No.: 14/353,130

(22) PCT Filed: Oct. 19, 2012
(86) PCT No.: PCT/EP2012/070816

§ 371 Date: Apr. 21, 2014

Related U.S. Application Data
(60) Provisional application No. 61/549,824, filed on Oct. 21, 2011.

(30) Foreign Application Priority Data
Oct. 21, 2011 (EP) 11306370.5

(51) Int. Cl. C07K 16/28

(52) U.S. Cl. CPC C07K 16/2866
USPC 530/389.6

(57) ABSTRACT

The invention relates to the prevention or the treatment of a condition associated with a chronic hyperactivation of the immune system, in particular to a M-DC8+ monocyte depleting agent for the prevention or treatment of chronic inflammatory or infectious diseases.

### FIELD OF THE INVENTION

The invention relates to the prevention or the treatment of a condition associated with a chronic hyperactivation of the immune system, in particular to a M-DC8+ monocyte depleting agent for the prevention or treatment of chronic inflammatory or infectious diseases.

### BACKGROUND OF THE INVENTION

HIV-1 infection induces the depletion of CD4+ T lymphocytes in the blood and the lymphoid organs, particularly in the gut-associated lymphoid tissue^{1,2}. In long-term non progressor or elite controller patients as well as in non-human primate models of HIV infection, pathogenicity has been correlated to chronic hyperactivation of the immune system^{3,4}. Systemic immune activation and progression of the disease were correlated to the increased translocation of gut luminal microbial products such as the gram-negative bacterial lipopolysaccharide (LPS)⁵. LPS stimulates the production of proinflammatory cytokines, and particularly TNFα. In HIV-1 infected patients, TNFα serum levels increase in correlation with disease progression and drop to normal levels following treatment only in patients with good virological and immunological responses⁶⁻⁸. By activating the NF-κB pathway, TNFα orchestrates chronic inflammation and immune activation, which drive the progression of the disease⁹. TNFα affects mucosal integrity, leading to microbial products systemic translocation, and it induces HIV replication in infected T cells¹⁰⁻¹⁵. Granulocyte/macrophage colony-stimulating factor (GM-CSF) and LPS also have an inductive effect on HIV replication in infected myeloid cells^{16,17}. GM-CSF and TNFα are mostly produced by monocytes and dendritic cells (DC) following LPS stimulation.

During chronic HIV infection, circulating plasmacytoid and myeloid dendritic cell (mDC and pDC) numbers are reduced¹⁸⁻²⁰. Myeloid DC were mostly studied in HIV-infected patients using CD11c as a marker. Now they are further subdivided into BDCA-1⁺ and BDCA-3⁺ mDC subsets, the latter recently shown as being the human homolog to mouse CD8α mDC²¹⁻²⁴. During HIV infection, circulating classical CD14⁺⁺CD16⁻ monocyte numbers are normal, but CD14^{+/-}CD16⁺⁺ monocyte numbers were found to be higher in HIV patients with advanced disease than in control donors^{25,26}. Interestingly, these cells are found in the brains from AIDS patients with HIV-related encephalitis and produce TNFα. Between these non-classical, CD14^{+/-}CD16⁺⁺ monocytes and the classical, CD14^{+/+}CD16⁻ monocytes, intermediate CD14⁺CD16⁺ monocytes can now be distinguished by sensitive multicolor flow cytometry^{27,28}. In addition, among CD14^{+/-}CD16^{+/+} monocytes, a subpopulation expressing M-DC8 [slan, 6-sulfo LacNAc, a glycosylation variant of P-selectin glycoprotein ligand-1 (PSGL-1)]²⁹ is proinflammatory and capable of stronger TNFα production following LPS stimulation than the other monocyte populations³⁰. These cells are found in abundance in inflamed tissues of patients with chronic inflammatory diseases such as Crohn's disease³¹ or psoriasis³², pathologies in which neutralizing anti-TNFα monoclonal antibodies are now the therapeutic gold standard.

However, such neutralizing anti-TNFα monoclonal antibodies may provoke an immunosuppression (which happens notably during HIV infection) leading to a risk of opportunistic infections. Thus, people taking such anti-TNFα antibodies are at increased risk for developing serious infections that may lead to hospitalization or death due to bacterial, mycobacterial, fungal, viral, parasitic, and other opportunistic pathogens.

So, there is a recognized and permanent need in the art for new reliable methods for preventing or treating conditions associated with a chronic hyperactivation of the immune system such as chronic inflammatory diseases and chronic infectious diseases, in particular chronic hyperactivation of the immune system during HIV infection.

### SUMMARY OF THE INVENTION

The invention is based on the discovery that M-DC8⁺ monocytes were mostly responsible for the strong LPS-induced TNFα overproduction in HIV-infected patients, and that these M-DC8⁺ monocytes can be depleted and/or induced to undergo apoptosis by the engagement of M-DC8, a glycosylation variant of P-selectin glycoprotein ligand-1 (PSGL-1). M-DC8⁺ monocytes depletion can be particularly useful for the prevention or the treatment of conditions associated with an excessive or unwanted immune response or excessive or unwanted TNFα productions such as chronic inflammatory diseases or infectious diseases (e.g. HIV infection). The invention thus relates to a M-DC8+ monocyte depleting agent for use in the prevention or treatment of a condition associated with a chronic hyperactivation of the immune system and more particularly a condition mediated by a TNFα overproduction such as chronic inflammatory diseases or infectious diseases (e.g. HIV infection).

### DETAILED DESCRIPTION OF THE INVENTION

### Definitions

Throughout the specification, several terms are employed and are defined in the following paragraphs.

The terms “M-DC8⁺ monocyte”, “M-DC8⁺ proinflammatory monocyte”, “M-DC8⁺ non-classical monocyte”, “TNFα-producing CD16⁺M-DC8⁺ cell”, “TNFα-producing MDC8⁺ cell”, “TNFα-producing MDC8⁺ monocyte”, “M-DC8⁺CD11c⁺CD14^{+/-}CD16⁺⁺ non-classical monocyte”, “CD16⁺M-DC8⁺ cell”, “CD16⁺⁺M-DC 8⁺ proinflammatory monocyte”, “M-DC8-expressing CD14^{+/-}CD16⁺⁺ monocyte”, “M-DC8+ macrophage”, “6-sulfo LacNAc-Positive Blood Dendritic Cell”, “slanDCs” and “slan cells” are used interchangeably herein to describe the particular kind of cell to be depleted in the context to the invention since such cell has been shown to be mostly responsible for the strong LPS-induced TNFα overproduction in HIV-infected patients and therefore for the chronic hyperactivation of the immune system notably in chronic infectious diseases (e.g. HIV infection). Thus, these terms refer to the pro-inflammatory monocyte population that produces TNF-α and other pro-inflammatory cytokines in response to microbial stimuli. It should be further reminded that these M-DC8⁺ monocytes are distinct from the CD14^{+/-}CD16⁺⁺ monocytes (CD14^{low}CD16^{high} monocytes).

A “M-DC8+ monocyte depleting agent” is a molecule which depletes or destroys MDC8+ monocytes in a patient and/or interferes with one or more M-DC8+ monocyte functions, e.g. by reducing or preventing TNF-α production by the M-DC8+ monocyte. The M-DC8+ monocyte depleting agent preferably binds to a M-DC8+ monocyte surface marker. The M-DC8+ depleting agent preferably is able to deplete M-DC8+ monocyte (i.e. reduce circulating M-DC8+ monocyte levels) in a patient treated therewith. Such depletion may be achieved via various mechanisms such as antibody-dependent cell mediated cytotoxicity (ADCC) and/or complement dependent cytotoxicity (CDC), inhibition of MDC8+ monocyte proliferation (e.g. via inhibition of generation of CD14⁺⁺CD16⁻ classical monocyte into MDC8+ monocyte) and/or induction of MDC8+ monocyte death (e.g. via apoptosis). MDC8+ monocyte depleting agents include but are not limited to antibodies, synthetic or native sequence peptides and small molecule antagonists which preferably bind to the M-DC8+ monocyte surface marker (preferably M-DC8), optionally conjugated with or fused to a cytotoxic agent. The preferred M-DC8+ monocyte depleting agent comprises an antibody, more preferably a M-DC8+ monocyte depleting antibody.

“Complement dependent cytotoxicity” or “CDC” refers to the lysis of a target cell in the presence of complement. Activation of the classical complement pathway is initiated by the binding of the first component of the complement system to antibodies which are bound to their cognate antigen. To assess complement activation, a CDC assay, e.g. as described in Gazzano-Santoro et al. (1997) may be performed.

“Antibody-dependent cell-mediated cytotoxicity” or “ADCC” refers to a form of cytotoxicity in which secreted antibodies bound onto Fc receptors (FcRs) present on certain cytotoxic cells (e.g. Natural Killer (NK) cells, neutrophils, monocytes and macrophages) enable these cytotoxic effector cells to bind specifically to an antigen-bearing target cell and subsequently kill the target cell. To assess ADCC activity of a molecule of interest, an in vitro ADCC assay, such as that described in U.S. Pat. No. 5,500,362 or 5,821,337 may be performed.

A “M-DC8+ monocyte surface marker” or “M-DC8+ monocyte target” or “M-DC8+ monocyte antigen” herein is an antigen expressed on the surface of a M-DC8+ monocyte which can be targeted with a M-DC8+ monocyte depleting agent which binds thereto. Exemplary M-DC8+ monocyte surface markers include but are not limited to the M-DC8 or other antigens that characterize the pro-inflammatory monocyte population that produces TNF-α and other pro-inflammatory cytokines in response to microbial stimuli.

The M-DC8+ monocyte surface marker of particular interest is preferentially expressed on M-DC8+ monocyte compared to other non-M-DC8+ monocyte tissues of a mammal. The terms “M-DC8” antigen and “slan” epitope are used interchangeably herein and refer to an O-linked sugar modification (6-sulfo LacNAc, slan) of P-selectin glycoprotein ligand-1 (PSGL-1). This antigen is characteristically expressed on a new subset of PBMCs with features closely related to CD14^{+/-}CD16⁺⁺ monocytes. Slan (M-DC8)+ cells constitute 0.5-2% of all PBMCs with similar frequencies among mononuclear cells from cord blood.

Examples of antibodies which bind the M-DC8 antigen that are contemplated by the invention include antibodies such as the anti-Slan (M-DC8) antibody (clone DD-1) which recognizes Slan (6-Sulfo LacNAc) purchased from Miltenyi Biotec under the reference 130-093-027 and the antibodies described in the international patent application published under n° WO 99/58678 included the antibody produced by hybridoma cell line DSM ACC2241 also called antibody M-DC8 (DC8). Said hybridoma cell has been deposited in the culture collection Deutsche Sammlung von Mikroorganismen and Zellkulturen GmbH (DSMZ) in Braunschweig, Germany on Oct. 26, 1995, in accordance with the Budapest Treaty. Other antibodies include those produced by hybridoma cell lines DSM ACC 2399 or DSM ACC 2998 described in the US patent application published under n° US 2007/0014798.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “chimeric antibody” refers to an antibody which comprises a VH domain and a VL domain of an antibody of the invention, and a CH domain and a CL domain of a human antibody. According to the invention, the term “humanized antibody” refers to an antibody having variable region framework and constant regions from a human antibody but retains the CDRs of the antibody of the invention.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH:: VL heterodimer which is usually expressed from a gene fusion including VH and VL encoding genes linked by a peptide-encoding linker. “dsFv” is a VH:: VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

“M-DC8+ monocyte depleting antibodies” are defined as those antibodies which bind to a M-DC8+ monocyte surface marker on the surface of M-DC8+ monocyte and mediate their destruction or depletion when they bind to said cell surface marker. The term includes antibody fragments and different antibody formats created from these fragments, in particular formats of chimerized or humanized, multispecific and/or multivalent antibodies. The “antibody formats” as referred to in the invention correspond to different combinations of domains and regions such as variable domains of heavy single chain antibodies (VHH) from Camelidae (camel, dromedary, llama), specifically recognizing a type of antigen.

The term “a condition associated with a chronic hyperactivation of the immune system” refers to a disorder or a disease associated with an excessive or unwanted immune response and more particularly a condition in which such excessive or unwanted immune response is mediated by a TNFα overproduction such as in chronic inflammatory diseases or in infectious diseases (e.g. HIV infection).

In the context of the invention, the term “treating” or “treatment”, as used herein, means reversing, alleviating, or inhibiting the progress of the disorder or condition to which such term applies, or one or more symptoms of such disorder or condition.

A “therapeutically effective amount” is intended for a minimal amount of active agent which is necessary to impart therapeutic benefit to a subject. For example, a “therapeutically effective amount” to a patient is such an amount which induces, ameliorates or otherwise causes an improvement in the pathological symptoms, disease progression or physiological conditions associated with or resistance to succumbing to a disorder. In its broadest meaning, the term “preventing” or “prevention” refers to preventing the disease or condition from occurring in a subject which has not yet been diagnosed as having it.

The term “patient” refers to any subject (preferably human) afflicted with or susceptible to be afflicted with.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

### Methods of Treatment

The present invention relates to a method for preventing or treating a condition associated with a chronic hyperactivation of the immune system in a patient in need thereof comprising the step of depleting the M-DC8+ monocytes population of said patient.

More particularly, the present invention relates to a method for preventing or treating a condition mediated by a TNFα overproduction in a patient in need thereof comprising the step of administrating said patient with a M-DC8+ monocyte depleting agent. The method according to the present invention can be supplied to a patient, which has been diagnosed as presenting a chronic inflammatory or infectious disease.

In a particular embodiment, said a chronic inflammatory disease is selected from the group consisting of rheumatoid arthritis, psoriasis, psoriatic arthritis, ankylosing spondylitis and inflammatory bowel disease (IBD) including ulcerative colitis, Crohn's disease and metabolic syndromes including atherosclerosis, obesity, diabetes and hypertension.

In a particular embodiment, said a chronic infectious disease is selected from the group consisting of HIV infection and other chronic viral diseases such as CMV, EBV and other herpes virus infections, HTLV-1 and other retroviral infections, and mycobacterial infections.

In a particular embodiment, the invention relates to a method for preventing or treating HIV infection in a patient in need thereof comprising the step of depleting the M-DC8+ monocytes population of said patient.

More particularly, the invention relates to a method for preventing or treating HIV infection comprising the step of administrating a patient in need thereof with a M-DC8+ monocyte depleting agent.

Preferably, the invention relates to a method for preventing or treating chronic hyperactivation of the immune system happening during the HIV infection comprising the step of administrating a patient in need thereof with a M-DC8+ monocyte depleting agent.

In particular embodiment the M-DC8+ monocyte depleting agent may consist in a M-DC8+ monocyte depleting antibody. Antibodies directed against a M-DC8+ monocyte surface marker can be raised according to known methods by administering the appropriate antigen or epitope to a host animal selected, e.g., from pigs, cows, horses, rabbits, goats, sheep, Camelidae (camel, dromedary, llama) and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique. Alternatively, techniques described for the production of single chain antibodies (see, e.g., U.S. Pat. No. 4,946,778) can be adapted to produce single chain antibodies against a M-DC8+ monocyte surface marker. Useful antibodies according to the invention also include antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to the M-DC8+ monocyte surface marker.

Humanized antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then after raising antibodies directed against a M-DC8+ monocyte surface marker as above described, the skilled man in the art can easily select those that deplete M-DC8+ monocytes, for example those that deplete M-DC8+ monocytes via antibody-dependent cell mediated cytotoxicity (ADCC), complement dependent cytotoxicity (CDC), inhibition of M-DC8+ monocyte generation or induction of M-DC8+ monocyte death (e.g. via apoptosis).

In a particular embodiment, the M-DC8+ monocyte depleting antibody may consist in an antibody directed against a M-DC8+ monocyte surface marker which is conjugated to a cytotoxic agent or a growth inhibitory agent. Such antibody may for instance one of those previously described in patent applications N° WO 99/58678 and N° US 2007/0014798.

Accordingly the invention contemplates the use of immunoconjugates comprising an antibody against a M-DC8+ monocyte surface marker conjugated to a cytotoxic agent or a growth inhibitory agent. A “growth inhibitory agent” when used herein refers to a compound or composition which inhibits growth of a cell, especially M-DC8+ monocyte, either in vitro or in vivo. Examples of growth inhibitory agents include agents that block cell cycle progression, such as agents that induce Gl arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest Gl also spill over into S-phase arrest, for example, DNA alkylating agents such as tamoxifen, prednisone, dacarbazine, mechlorethamine, cisplatin, methotrexate, and 5-fluorouracil.

The term “cytotoxic agent” as used herein refers to a substance that inhibits or prevents the function of cells and/or causes destruction of cells. The term is intended to include radioactive isotopes (e.g. At211, 1131, 1125, Y90, Rel86, Rel88, Sml53, Bi212, P32, and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleo lytic enzymes, antibiotics, and toxins such as small molecule toxins or enzymatically active toxins of bacterial, fungal, plant or animal origin, including fragments and/or variants thereof, e.g., gelonin, ricin, saporin, and the various antitumor or anticancer agents disclosed below. Other cytotoxic agents are described below. A tumoricidal agent causes destruction of tumor cells.

Conjugation of the antibodies of the invention with cytotoxic agents or growth inhibitory agents may be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6 diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radio nucleotide to the antibody (WO 94/11026).

Alternatively, a fusion protein comprising the antibody and cytotoxic agent or growth inhibitory agent may be made, by recombinant techniques or peptide synthesis. The length of

DNA may comprise respective regions encoding the two portions of the conjugate either adjacent one another or separated by a region encoding a linker peptide which does not destroy the desired properties of the conjugate.

In a particular embodiment, the preferred M-DC8+ monocyte surface marker is M-DC8.

Thus, in a preferred embodiment of the invention, M-DC8+ monocyte depleting agent is an anti-M-DC8 antibody.

Preferably, said M-DC8+ monocyte depleting agent is administered in a therapeutically effective amount. By a “therapeutically effective amount” is meant a sufficient amount of the M-DC8+ monocyte depleting agent to treat or to prevent a condition associated with a chronic hyperactivation of the immune system at a reasonable benefit/risk ratio applicable to any medical treatment.

In another embodiment the M-DC8+ monocyte depleting agent may consist in an agent reducing or inhibiting the generation of MDC8+ monocytes from CD14++CD16-classical monocytes.

Preferably, said agent is an antagonist of the GM-CSF receptor (GM-CSFR) or the M-CSF receptor (M-CSFR) or a combination thereof.

By “receptor antagonist” is meant a natural or synthetic compound that has a biological effect opposite to that of a receptor agonist. The term is used indifferently to denote a “true” antagonist and an inverse agonist of a receptor. A “true” receptor antagonist is a compound which binds the receptor and blocks the biological activation of the receptor, and thereby the action of the receptor agonist, for example, by competing with the agonist for said receptor. An inverse agonist is a compound which binds to the same receptor as the agonist but exerts the opposite effect. Inverse agonists have the ability to decrease the constitutive level of receptor activation in the absence of an agonist.

The terms “M-CSF receptor antagonist” or “GM-CSF receptor antagonist” include any entity that, upon administration to a patient, results in inhibition or down-regulation of a biological activity associated with activation of the receptor by their natural ligand, respectively M-CSF or GM-CSF in the patient, including any of the downstream biological effects otherwise resulting from the binding to the receptor with their natural ligand. Such receptor antagonists include any agent that can block M-CSF or GM-CSF receptor activation or any of the downstream biological effects of M-CSF or GM-CSF receptor activation. For example, such a M-CSF or GM-CSF receptor antagonist (e.g. a small organic molecule, an antibody directed against M-CSF or GM-CSF) can act by occupying the ligand binding site or a portion thereof of the M-CSF or GM-CSF receptor, thereby making these receptors inaccessible to their natural ligands, M-CSF or GM-CSF, so that its normal biological activity is prevented or reduced. The terms M-CSF or GM-CSF receptor antagonist include also any agent able to interact with the natural ligand, namely M-CSF or GM-CSF. Said agent may be an antibody directed against M-CSF or GM-CSF which can block the interaction between M-CSF or GM-CSF and their respective receptor or which can block the activity of M-CSF or GM-CSF (“neutralizing antibody”).

The term “blocking the interaction”, “inhibiting the interaction” or “inhibitor of the interaction” are used herein to mean preventing or reducing the direct or indirect association of one or more molecules, peptides, proteins, enzymes or receptors; or preventing or reducing the normal activity of one or more molecules, peptides, proteins, enzymes, or receptors.

Such M-CSF receptor antagonists and GM-CSF receptor antagonists are well known in the art. Examples of M-CSF receptor antagonists that are contemplated by the invention include antibodies which bind the M-CSF such as the monoclonal antibody 5H4 (ATCC Accession No. HB 10027) described in the international patent application N° WO 2004/045532. Examples of GM-CSF receptor antagonist that are contemplated by the invention include antibodies which bind the anti-GM-CSF such as monoclonal antibodies described in the international patent application N° WO 2010093814.

Alternatively, said agent reducing or inhibiting the generation of MDC8+ monocytes from CD14++CD16− classical monocytes may be IL4, IL10 or a combination thereof.

Interleukin 4 (IL4) and Interleukin 10 (IL10) have their general meaning in the art.

The naturally occurring human IL4 protein has an amino acid sequence shown in Genbank, Accession number NP_{—}000580.1 and the naturally occurring human IL10 protein has an amino acid sequence shown in Genbank, Accession number NP_{—}000563.1.

Within the context of the invention, it is intended that IL4 and IL10 derivatives are encompassed. As used herein, a IL4 and IL10 derivatives encompasses IL4 variants and fragments as well as IL10 variants and fragments.

As used herein, a “IL4 variant” encompasses polypeptides having at least about 80 percent, or at least about 85, 90, 95, 97 or 99 percent sequence identity with the sequence of human IL4. As used herein, a “IL10 variant” encompasses polypeptides having at least about 80 percent, or at least about 85, 90, 95, 97 or 99 percent sequence identity with the sequence of human IL10. As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously align according to the best alignment; this comparison is realized on segments of comparison in order to identify and compared the local regions of similarity. The best sequences alignment to perform comparison can be realized, beside by using for example computer softwares using such algorithms (GAP, BESTFIT, BLAST P, BLAST N, FASTA, TFASTA). To get the best local alignment, one can preferably used BLAST software, with the BLOSUM 62 matrix, or the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences. It will also be understood that natural amino acids may be replaced by chemically modified amino acids. Typically, such chemically modified amino acids enable to increase the polypeptide half life.

As used herein, a “IL4 fragment” is a biologically active portion of IL4 polypeptide. A “biologically active” portion of IL4 polypeptide includes a IL4-derived peptide that possesses one or more of biological activities of IL4.

As used herein, a “IL10 fragment” is a biologically active portion of IL10 polypeptide. A “biologically active” portion of IL10 polypeptide includes a IL10-derived peptide that possesses one or more of biological activities of IL10.

Methods for producing recombinant proteins are known in the art. The skilled person can readily, from the knowledge of a given protein's sequence or of the nucleotide sequence encoding said protein, produce said protein using standard molecular biology and biochemistry techniques.

It will be understood that the total periodically usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Compositions

The M-DC8+ monocyte depleting agent of the invention may be combined with pharmaceutically acceptable excipients, and optionally sustained-release matrices, such as biodegradable polymers, to form therapeutic compositions.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The M-DC8+ monocyte depleting agent of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The M-DC8+ monocyte depleting agent of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: M-DC8+ non-classical monocytes from untreated HIV-infected patients produce greater amounts of TNFα than those from healthy donors and are the major source of TNFα following LPS stimulation: (a) TNFα plasmatic concentrations from 16 healthy donors (open circles), 8 HIV-infected, treated (grey circles) and 15 untreated (filled circles) patients. (b-d) Following 18 h stimulation with or without LPS, TNFα concentrations were measured in culture supernatants from (b) total PBMC (8 healthy donors and 7 untreated HIV-infected patients), (c) total vs. [M-DC8⁺ cell]-depleted PBMC (4 healthy donors and 3 HIV-infected, untreated patients), (d) FACS-sorted M-DC8⁺ non-classical monocytes from 4 healthy donors and 4 untreated patients, and (e) monocyte subsets from one healthy blood donor (representative of three independent experiments).

FIG. 2: Summary of mechanisms underlying the strong increase in CD16⁺⁺M-DC8⁺ proinflammatory monocytes that could account for TNFα-mediated chronic inflammation, a hallmark of HIV-infection: Chronic immune activation drives the progression of HIV-infection and is thought to be the best prediction parameter of disease outcome. As in Crohn's disease, such activation seems to be predominantly driven by systemic LPS translocation and TNFα overproduction, a pillar of chronic inflammation. However the cellular origins of this TNFα overproduction has remained elusive. We demonstrate here that in the blood from HIV-infected, untreated patients, CD16⁺⁺M-DC8⁺ proinflammatory monocytes recapitulate the TNFα overproduction and can arise in vitro from CD14⁺⁺CD16⁻ classical monocytes in a proinflammatory environment, (GM-CSF) two major events implicated in the physiopathlogy of LPS-driven HIV-disease progression. Also, it was previously published that GM-CSF gene expression is induced following the activation of the NF-κB pathway, that is activated by both LPS and TNFα{Pomerantz, 1990 #46; Shannon, 1997 #50}.

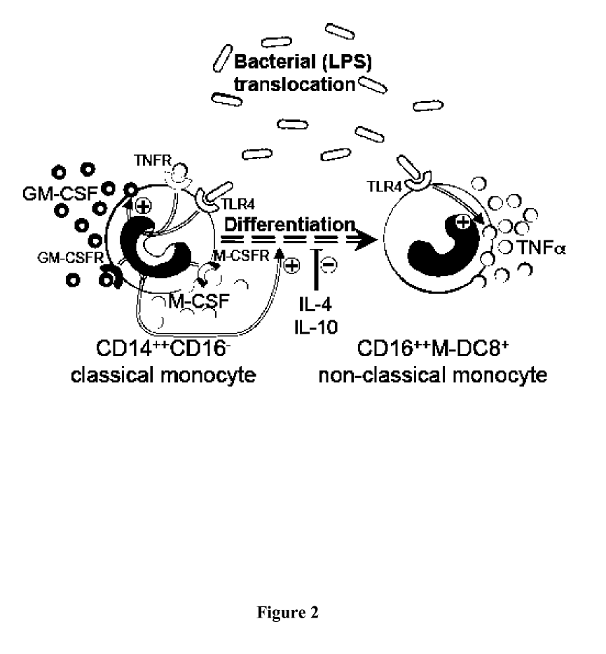

### EXAMPLE

### Example 1

### Pivotal Role of M-DC8+ Monocytes from Viremic HIV Infected Patients in TNFα Over-Production in Response to Microbial Products

Material & Methods

Patient Samples:

Peripheral blood was collected on heparin from 23 patients with chronic HIV-1 infection, included in the prospective cohorts PREVAC (Clinical Investigation center of the Cochin Hospital, Paris) and PRIMO-ANRS (Table 1). This study was approved by the Comité de Protection des Personnes dans la Recherche Biomédicale (Paris, France) and all patients gave an informed consent before inclusion. The human study was conducted according to the principles expressed in the Declaration of Helsinki. Patients were aged 20-64 years (median: 39 years). Fifteen were treated by combined antiretroviral therapy (cART) and 8 were untreated. Untreated patient's VLs ranged from 1.63 to 4.98 Log₁₀ HIV RNA copies/ml (median: 4.25 Log₁₀ copies of HIV RNA/ml) and their CD4⁺ T cell counts from 279 to 803 cells/μl (median: 544 cells/μl). For comparison, peripheral blood from 16 uninfected individuals was collected on heparin at the Etablissement Français du Sang of the Saint-Vincent de Paul Hospital (Paris, France) within an ethics convention with INSERM. All experiments were carried out with PBMC freshly purified on a Ficoll density gradient. Plasma (diluted 1:1 with NaCl) were isolated from the top layer of the Ficoll gradient and frozen.

Spleen samples originated from 6 HIV-infected and 4 uninfected patients requiring therapeutic or diagnostic splenectomy (Idiopathic Thrombopenic Purpura, adherence to pancreatic cancer, . . . ), were collected with informed consent obtained in accordance with the Declaration of Helsinki and with approval from the Comité de Protection des Personnes Ile de France III, Institutional Review Board for these studies (Table 1), and prepared as previously described⁵⁹. Blocks of spleen were cut into small pieces, forced through a sterile sieve mesh, and cells dissociated with type VII collagenase, DNase I (20 U/ml; Sigma-Aldrich) and 10 mM ethylenediaminetetraacetic acid. Surface molecule expression was not affected by this enzymatic dissociation⁶⁰. Spleen mononuclear cells (SMC) were isolated from splenocyte suspensions on a Ficoll density gradient and immediately frozen. Cells were all thawed prior to flow cytometric analyses.

11-Color Flow Cytometric Analyses and Intracellular TNFα Detection:

The following monoclonal antibodies were used in this study: For 11-color membrane flow cytometric analyses: M-DC8-FITC (clone DD-1, dilution factor: 1/20), CD141(BDCA-3)-APC (clone AD5-14H12, 1/150) and CD303(BDCA-2)-PE (clone AC144, 1/10) from Miltenyi Biotec; CD1c(BDCA-1)-Pacific Blue (clone L161, 1/400; Biolegend); CD14-QDot655 (clone TüK4, 1/100; Invitrogen), CD19-ECD (clone J3-119, 1/10; Beckman Coulter), CD11c-AlexaFluor700 (clone 3.9, 1/10; eBioscience); HLA-DR-PerCP (clone G46-6, 1/10), CD16-APC-H7 (clone 3G8, 1/40) and CD45-Amcyan (clone 2D1, 1/25) from BD Biosciences. For intracellular cytokine expression analyses and for FACS-sorting of monocyte subsets: CD141(BDCA-3)-PE (clone AC144, 1/10; Miltenyi Biotec); HLA-DR-ECD (clone Immu-357, 1/10; Beckman Coulter); CD19-APC-H7 (clone SJ25C1, 1/15), CD14-PE-Cy7 (clone M5E2, 1/30) and TNFα-AlexaFluor700 (clone MAB11, 1/20) from BD Biosciences. After 4-day cultures of FACS-sorted classical CD14⁺CD16⁻M-DC8⁻ monocytes: CD1a-PE (clone HI149, 1/10; BD Biosciences) was also used. In all experiments, the Live/Dead blue Dye (Invitrogen) was used to exclude dead cells.

For 11-color membrane and 9-color intracellular FACS analyses, freshly purified PBMC (2.10⁶ cells/tube) were used, and in the later experiments, PBMC were stimulated for 7 h at 37° C., with 5% CO₂, in polypropylene tubes in complete RPMI 1640 supplemented with 10% FCS with or without lipopolysaccharide at 100 ng/ml (LPS; Sigma). Brefeldin A (BFA; Sigma) was added for the last four hours at a final concentration of 10 μg/ml. PBMC were washed and incubated for 30 min at +4° C. with Live/Dead blue dye in PBS. 5% decomplemented AB human serum (serum-AB, Abcys) was added for an extra 15 min at +4° C. Next, cells were labeled for 30 min at +4° C. with antibodies diluted in PBS with 2% FCS and 2 mM EDTA. For intracellular FACS-analyses, cells were fixed and permeabilized with BD Cytofix/Cytoperm kit (BD Biosciences) following manufacturer's instructions and incubated with the anti-TNFα monoclonal antibody (45 min, +4° C.). Cells were then washed, fixed with 0,5% paraformaldehyde and events acquired using a BD FACS LSRII (BD Biosciences). All analyses were carried out with the BD FACSDiva (BD Biosciences) software. The median number of analyzed events for the CD141(BDCA-3)⁺ dendritic cell population was 188, the minimum was 17 and the highest was 5927. Other DC and monocyte subsets were more numerous. The absolute number of cells/blood μl was calculated by multiplying the hemocytometer complete blood count of mononuclear cells (monocytes+lymphocytes) to the percentage of cells among CD45^{hi} events.

Flow Cytometry Cell Sorting:

Freshly purified PBMC were incubated for 15 min at +4° C. with 5% decomplemeted serum-AB in PBS and labeled with the following antibodies prior to FACS-sorting using a BD FACSAriaIII (BD Biosciences) set for high purity sorting. Purified cells were at least 98% pure. For the 4 HLA-DR⁺CD11c⁺ monocyte subsets sorting, cells were labeled with the following antibodies: M-DC8-FITC, HLA-DR-PerCP, CD14-PE-Cy7, CD16-APC-H7 and CD11c-AlexaFluor700. For depletion of CD11c⁺M-DC8⁺ non-classical monocytes from PBMC, M-DC8-FITC was used alone to leave sorted cells untouched.

In Vitro Monocyte Differentiation:

Freshly FACS-sorted classical HLA-DR⁺CD11c⁺CD14^{hi}CD16⁻M-DC8⁻ monocytes were cultured for 4 days in RPMI 1640 supplemented with 10% FCS and cultured at 37° C. with 5% CO₂ in the presence or not of GM-CSF (50 ng/ml, AbCys) and M-CSF (10 ng/ml, AbCys) in flat-bottom 96 well-plates. When indicated, IL-4 (200 UI/ml, AbCys) or IL-10 (10 ng/ml, R&D Systems) were added. Cells were then thoroughly recovered with ice-cold PBS containing 2 mM EDTA without leaving any remaining adherent cell in the wells prior to either LPS stimulation for intracellular TNFα expression assessment or direct FACS staining as described above using the following antibodies: M-DC8-FITC, CD11c-AlexaFluor700, HLA-DR-PerCP, CD14-PE-Cy7, CD16-APC-H7 and CD1a-PE.

Cytokines Concentration Measurement:

Total PBMC (2.10⁶ cells in 500 μl or 1.10⁵ cells in 100 μl for M-DC8 depletion experiments), FACS-sorted monocyte subsets (5.10⁴ cells in 100 μl), were cultured in RPMI 1640 supplemented with 10% FCS at 37° C. with 5% CO₂ in the presence or not of LPS for 18 h. Supernatants were collected after centrifugation and stored at −80° C. until use. For the quantification of TNFα and GM-CSF, Cytometric Beads Arrays (BD Biosciences) were used following the manufacturer's instructions (Flow cytometric beads were analyzed with a BD LSRII flow cytometer). Concentrations were determined using the FCAP Array software (BD Biosciences). TNFα and GM-CSF in plasma diluted 1:1 in NaCl were quantified using the FCAP Array software (BD Biosciences). TNFα and GM-CSF in plasma diluted 1:1 in NaCl were quantified using highly sensitive quantikine ELISA kits (R&D systems).

Statistical Analysis:

Results are given as medians. The Mann-Whitney test was used to compare controls and patients or cellular subsets. Correlations were evaluated with the Spearman test. Differences were defined as statistically significant when p<0.05. All these non-parametric tests were performed using the GraphPad Prism 5 software.

Results

Depletion of Dendritic Cells and Expansion of CD16+ Monocytes in the Blood and Spleens from Viremic, HIV-Infected, Untreated Patients:

In order to study all dendritic cell and monocyte subsets simultaneously, we carried out 11-color flow cytometric analyses. Peripheral blood mononuclear cells (PBMC) from 13 healthy blood donors, 8 HIV-infected patients treated by combined antiretroviral therapy (cART) and therefore aviremic (named “virally suppressed HIV-infected patients”), and 15 HIV-infected, untreated patients (named “HIV-infected patients”) and spleen mononuclear cells (SMC) from 6 HIV-infected and 4 uninfected patients were studied (Table 1). The gating strategy used to separate the various cellular subsets is shown for representative uninfected individuals. In these analyses, CD45^{hi}HLA-DR⁺CD19⁻ cells were subdivided into three dendritic cell-subsets [CD303(BDCA-2)⁺ plasmacytoid DC (pDC), CD141(BDCA-3)⁺ and CD1c(BDCA-1)⁺ myeloid DC (mDC)], and three major monocyte subsets (CD14⁺⁺CD16⁻ classical, CD14⁺CD16⁺ intermediate and CD14^{+/-}CD16⁺⁺ non-classical monocytes). Non-classical monocytes were further subdivided based on the expression of M-DC8. Dot plots defining DC and monocyte subsets in blood and spleen from representative HIV-infected and uninfected individuals are shown.

The absolute numbers and proportions of circulating BDCA-3⁺ mDC, shown recently to be the human equivalents of the mouse CD8α⁺ mDC population, were reduced in HIV-infected patients (Table 1), compared to healthy controls (556±332 vs. 1096±1457 cells/ml, p=0.0003; 0.02±0.01% vs. 0.06±0.04% among CD45+PBMC, p=0.0003). The absolute numbers and proportions of circulating BDCA-1⁺ mDC, and pDC, labeled by BDCA-2-specific antibodies, were also reduced in HIV-infected patients as compared to healthy controls (BDCA-1⁺ mDC: 6112±3348 vs. 9928±5791 cells/ml, p=0.006; 0.22±0.18% vs. 0.51±0.17%, p=0.0008, and BDCA-2⁺ pDC: 4787±3856 vs. 9768±8426 cells/ml, p=0.02; 0.18±0.16% vs. 0.47±0.23%, p=0.004). The numbers and proportions of all DC subsets in the virally suppressed HIV-infected patients were not statistically different from those of the controls.

Interestingly, in the spleens from HIV-infected patients, the proportions of both mDC subsets were strongly reduced as compared to uninfected patients, particularly those of BDCA-3⁺ mDC, with a median proportion reduced almost 10 times (BDCA-3⁺ mDC: 0.03±0.06% vs. 0.29±0.11%, p=0.01; and BDCA-1⁺ mDC: 0.15±0.15% vs. 0.94±0.39%, p=0.01). In the spleen, BDCA-2⁺ pDC proportions were not different between HIV-infected and uninfected patients (0.31±0.34% vs. 0.26±0.07%).

We next addressed monocyte subsets in the blood. The median numbers and percentages among CD45^{hi}PBMC of both CD16⁺ subsets, but not of classical CD14⁺⁺CD16⁻ monocytes, were higher in HIV-infected patients as compared to healthy donors. The monocytes with the highest CD16 expression were the most increased (CD14^{+/-}CD16⁺⁺ non-classical monocytes: 35.7±27.3.10³ vs. 13.7±10.7×10³ cells/ml blood, p=0.0009; 1.23±1.46% vs. 0.70±0.54%, p=0.008; and CD14⁺CD16⁺ intermediate monocytes: 22.3±15.7×10³ vs. 10.2±9.4×10³ cells/ml blood, p=0.008; 0.97±0.50% vs. 0.49±0.47%, p=0.02). Virally suppressed HIV-infected patients had similar numbers of all monocyte subsets as compared to control donors.

In the spleens from HIV-infected patients, the proportions of both CD16⁺ monocyte subsets were also strongly higher than those from uninfected patients (0.45±1.32% vs. 0.09±0.07%, p=0.02 for intermediate and 0.49±2.14% vs. 0.13±0.06%, p=0.01 for non-classical monocytes).

The M-DC8+ Subset Mostly Accounts for the High Numbers of Blood and Spleen Non-Classical CD14loCD16++ Monocytes:

Non-classical CD14^{lo}CD16⁺⁺ monocytes can be subdivided into CD11c-MDC8−, CD11c⁺M-DC8⁻ and CD11c⁺M-DC8⁺ subsets. M-DC8⁺ non-classical monocytes median numbers and percentages among CD45^{hi} PBMC were strongly increased in HIV-infected patients as compared to healthy donors (23.6×10³±26.1×10³ vs. 8.4×10³±6.7×10³ cells/ml blood, p=0.0002; 0.83±1.35% vs. 0.41±0.36%, p=0.003) and virally suppressed HIV-infected patients (23.6×10³±26.1×10³ vs. 9.4×10³±6.3×10³ cells/ml blood, p=0.003; 0.83±1.35% vs. 0.45±0.27%, p=0.03). This was also the case in the spleen (0.31±1.17% vs. 0.06±0.03%, p=0.01;). In these patients, the proportion of M-DC8⁺ cells was increased among total non-classical CD14^{+/-}CD16⁺⁺ monocytes as compared to healthy individuals (75% vs. 55% in the blood; 60% vs. 43% in the spleen). M-DC8⁻ non-classical monocyte numbers and percentages were not significantly different in the blood and spleens of HIV patients and healthy donors. Conversely, the increased numbers of MDC-8⁺ cells in these patients accounted for the increased numbers of non-classical monocytes (Spearman r=0.97, p<0.0001).

M-DC8+ Non-Classical Monocytes are Responsible for the LPS-Induced TNFα-Overproduction in HIV-1-Infected, Untreated Patients:

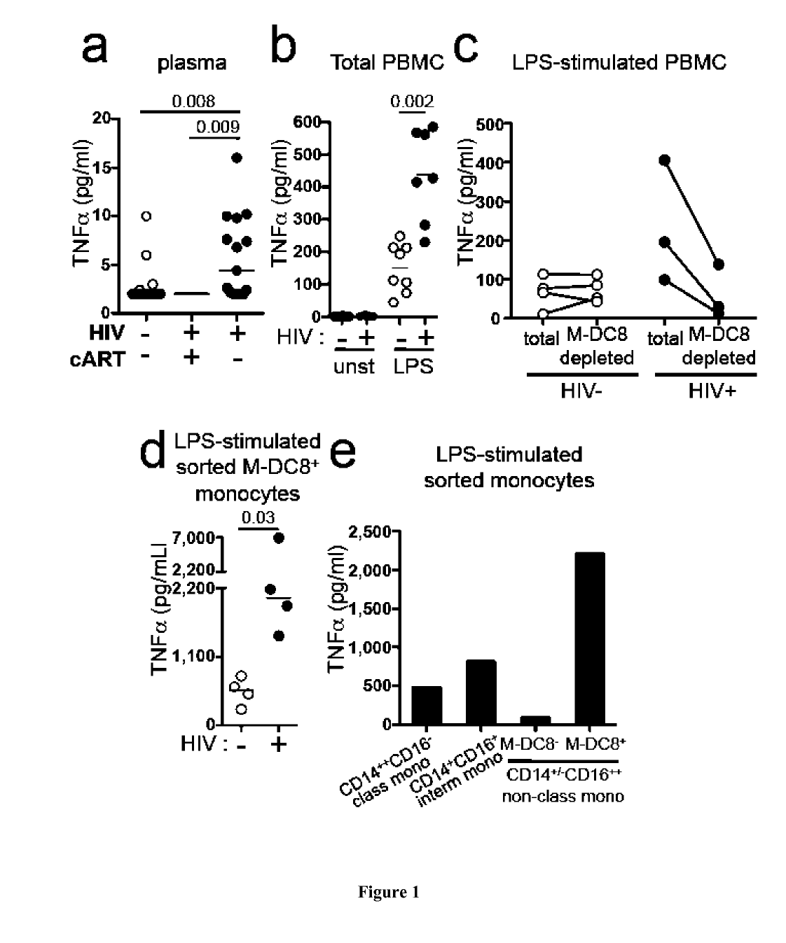

TNFα plasmatic concentrations, were significantly increased in plasma from HIV-infected patients as compared to both healthy donors (p=0.008) and virally suppressed HIV-infected patients (p=0.009; FIG. 1a). To assess the role of the different myeloid cell populations in TNFα production, we first cultured freshly purified PBMC from 8 healthy blood donors and 7 HIV-infected patients for 18 hours in the presence of LPS (FIG. 1b). While no TNFα could be detected in the supernatants from unstimulated PBMC, there was a strongly increased TNFα production by LPS-stimulated PBMC from HIV-infected patients as compared to healthy donors (p=0.002). Next, to determine the contribution of M-DC8⁺ non-classical monocytes to the total TNFα production by LPS-stimulated PBMC, M-DC8-expressing cells were depleted by FACS-sorting from the PBMC of 4 healthy donors and 3 HIV-1-infected patients (FIG. 1c). While M-DC8-depletion did not apparently affect LPS-induced TNFα production from healthy donors, it induced a mean 6-fold drop in TNFα production (individual from the HIV-infected patients: 8.1, 6.7 and 2.9 fold). Furthermore, TNFα production by M-DC8-depleted PBMC from HIV-infected patients reached a level comparable to that observed for healthy donor PBMC. Also, following LPS stimulation, FACS-sorted M-DC8⁺ non-classical monocytes from HIV infected patients showed a 3.6 fold increase in TNFα production as compared to healthy donors (p=0.03, FIG. 1d), and were also the strongest TNFα-producing monocyte subset (FIG. 1e). In order to assess the production of TNFα by DC and monocyte subsets from a greater number of donors and HIV-infected patients, TNFα intracellular FACS analyses were carried out using freshly purified PBMC. Of note, monocytes downregulated CD16 expression following culture and could therefore not be defined on the basis of CD16 expression. The two mDC subsets produced moderate levels of TNFα, that were not significantly different between donors and infected patients, while B lymphocytes and CD19⁻ cells falling in the lymphocyte gate (mostly T and NK cells) did not produce any TNFα. While the median percentage of TNFα-positive CD14^{hi} and CD14^{lo}M-DC8⁻ monocyte subsets were only moderately increased in HIV-infected patients following LPS stimulation (p=0.04 and p=0.02, respectively), their was a strong increase in the percentage of TNFα-positive M-DC8⁺ monocytes as compared to controls following LPS stimulation (p=0.003). Furthermore, the median percentage of TNFα-positive M-DC8⁺ monocytes was much higher than that of both CD14^{hi} and CD14^{lo}M-DC8⁻ monocytes from HIV-infected patients (86.7% vs. 42.7%, p=0.002 and vs. 31.2%, p=0.0002, respectively; FIG. 3h). M-DC8⁺ monocytes from HIV-infected patients not only had a greater percentage of TNFα-positive cells but showed also a much greater MFI of the TNFα-positive population as compared to both CD14^{hi} (p=0.0006) and CD14^{lo}M-DC8⁻ (p=0.001) monocytes and to M-DC8⁺ monocytes from control donors (p=0.02).

CD16+M-DC8+ Cells Differentiate from Classical CD14++CD16-M-DC8− Monocytes Under Inflammatory Conditions In Vitro:

In order to understand why M-DC8⁺ non-classical monocytes counts were higher in the blood from HIV-infected patients, we correlated them to those of other cellular subsets, and observed a significant inverse correlation with CD14⁺⁺CD16⁻ classical monocyte counts (Spearman r=−0.61, p=0.016). This was not the case for the counts of the other monocyte subsets. This inverse correlation led us to raise the hypothesis that M-DC8⁺ non-classical monocytes might differentiate from CD14⁺⁺CD16⁻ classical monocytes. FACS-sorted CD14⁺⁺CD16⁻MDC-8⁻ classical monocytes from two HIV-infected patients and three healthy blood donors were cultured in the presence of GM-CSF and M-CSF. After 4 days of culture, CD16 and M-DC8 expression were acquired by a large proportion of cells, (9.7-39.4% of M-DC8⁺ cells) for the 5 individuals tested, whether they were infected by HIV or not. This differentiation was not associated with the expression of the monocyte-derived dendritic cell (MDDC) CD1a antigen, which is induced by culture with IL-4^{33,34}. Most interestingly, the addition of both IL-4 and IL-10 both strongly inhibited the differentiation into M-DC8-expressing cells, whereas IL-4 induced an increase in CD1a expression as expected. One explanation for the increase of M-DC8⁺ monocytes in HIV-infected patients could be linked to the strong immune activation that occurs during HIV-1 infection. Indeed, we found, as previously published, increased GM-CSF concentrations in the plasma from HIV-infected patients (n=15) as compared to both healthy donors (n=16; p=0.03) and virally suppressed HIV-infected patients (n=8, p=0.05). We also observed a stronger capacity of both total PBMC (p=0.04) and FACS-sorted CD14⁺⁺CD16⁻ classical monocytes from HIV-infected patients (n=3) to produce GM-CSF as compared to cells from healthy donors (n=4). Thus, the proinflammatory cytokine environment including the GM-CSF measured here in the plasma from chronically infected patients, may be responsible for the increased proportion and count of pro-inflammatory M-DC8⁺ monocytes. Finally, we could also observe that after 4 days of culture of primary CD14⁺⁺CD16⁻ monocytes with GM-CSF and M-CSF, following LPS stimulation, the strongest TNFα production was observed in M-DC8⁺ cells that, following activation, had downregulated their CD16 expression.

### Example 2

### Localization and Quantification of M-DC8+ Monocytes on Spleen Cryosections from Patients

Material and Methods:

Localization and quantification of M-DC8+ monocytes were performed by immunohistofluorescence on spleen cryosections from 17 patients (8 uninfected, 9 HIV-infected). 7 μm spleen cryosections were blocked, incubated with primary antibodies (M-DC8 DD2, a kind gift from Pr K. Schäkel (University of Heidelberg), CD11c, CD68, ASM) and then with secondary antibodies. Nuclei were counterstained with DAPI. Sections were analyzed with an Observer Z.1 Zeiss microscope (Carl Zeiss) equipped with an Orca ER camera (Cochin Imaging Facility). Acquisitions were done under a x40 1.6 oil objective and using the Metamorph “Virtual slide” module where 5×5 assembled images were performed giving rise to a total of 0.69 mm² tissue area. Image analyses were done using Image J software. Statistical analysis (Mann-Whitney) was performed using GraphPad Prism software.

Results:

M-DC8+ cells showed the same labelling pattern in situ than after ex vivo isolation. In situ M-DC8+ cells were also CD11c+ and CD68+, as bona fide monocyte/macrophages. The numbers of M-DC8+ cells were higher in HIV-infected patients than in uninfected patients. Moreover, in situ labeling showed that if M-DC8+ cells were localized in the red pulps from all patients, they were present within the marginal zone only in HIV-infected, untreated patients.

Discussion

These results point to MDC8⁺ proinflammatory monocytes as a major myeloid cell population that is not depleted, but expanded during HIV chronic infection in the absence of viral load control. Here, using an 11-color flow cytometric strategy, we found high CD16⁺ monocyte cell counts in asymptomatic, chronically infected patients, as had previously been shown only in patients with AIDS or AIDS-related dementia^{25,26,35}.³⁶. Furthermore, we pointed to the M-DC8⁺ subset, which plays a role in several inflammatory diseases but had never been studied in HIV-1 infected patients, as the main responsible for this elevation. We also found normal counts in patients whose viral loads were controlled by cART, indicating restoration by treatment, but this needs to be confirmed in prospective studies.

One hypothesis to explain this increase in circulating M-DC8⁺ monocyte counts would be a defective migration into tissues. This seems unlikely since these cells infiltrate inflamed tissues in chronic inflammatory diseases^{31,32} Also, CD16⁺ monocytes infiltrate the brains from patients with AIDS-related dementia^{37,38}. Finally, the proportion of CD16⁺ monocytes, and especially M-DC8⁺ monocytes, is very high in spleens from the HIV-infected patients studied here compared to uninfected controls. A second hypothesis would be chemotaxis, like for brain infiltration in AIDS patients, where these CX3CR1-positive cells can be attracted by the high levels of CX3CL1 detected in the brain from these patients^{37,39-41} and induced in astrocytes by TNFα⁴². Further histological studies are needed to assess the chemokine and chemokine receptor expressions in these spleens. A third hypothesis would be a greater differentiation of classical monocytes into M-DC8⁺ cells. In the presence of GM-CSF and MCSF, FACS-sorted primary CD14⁺⁺CD16⁻ monocytes acquired both CD16 and M-DC8 expression together with a greater TNFα-production capacity following LPS stimulation. This was not the case in the presence of IL-10 or IL-4, in accordance with others⁴³. Indeed, these two cytokines rather favor an M2 or DC-like polarization of monocytes in vitro, whereas LPS, TNFα and GM-CSF cooperate to induce a proinflammatory M1 polarization that is associated to a strong TNFα production by polarized cells⁴⁴. Furthermore, activation of the NF-κB pathway, which is mediated by both LPS or TNFα, induces GM-CSF gene expression^{17,45}, while M-CSF, which is found at high concentrations in healthy human blood⁴⁶, is also synergistically induced by GM-CS F and TNFα⁴⁷. This differentiation may really have happened in vivo in the HIV-infected, untreated patient group for the following reasons. a) These patients displayed an inverse correlation between classical CD14⁺⁺CD16⁻ and CD14^{+/-} CD16⁺⁺M-DC8⁺ monocyte counts; b) they also had significantly higher plasmatic levels of TNFα, and of GM-CSF. T lymphocytes or NK lymphocytes may also participate in TNFα production, but not directly in response to LPS (as confirmed in our experiments in vitro, not shown).

In this study we also characterized a major functional consequence of the increase in proinflammatory M-DC8⁺ monocytes, showing that among PBMC and among other, they are responsible for the overproduction of TNFα in vitro in response to LPS in the blood from HIV-infected, untreated patients. This is also really likely to happen in vivo, as plasmatic TNFα levels were higher than normal in these patients, as expected^{6-8,48,49}. TNFα also induces HIV-1 replication in CD4⁺ T lymphocytes^{14,16}. In AIDS-related dementia, high TNFα levels are also found in the spinal fluid, opening the way for HIV-1 invasion of CD16⁺ monocytes from the blood to the brain^{50,51}, and cognitive dysfunction correlates with high plasmatic levels of soluble TNFRII (which at physiological concentrations stabilizes the bioactivity of TNFα⁵²), CD14 and LPS^{36,53}. In Crohn's disease, M-DC8⁺ cells are found in abundance in inflamed mucosal tissues³¹, and they produce large amounts of TNFα, which is a central actor of the intestinal epithelial cells destruction leading to LPS translocation^{10,11,13,54}. Like in Crohn's disease, TNFα-producing M-DC8⁺ cells in the mucosa from HIV-infected patients may have a major role in the maintenance of chronic immune activation leading to the strong mucosal CD4⁺ T lymphocyte depletion⁵.

In former studies during HIV infection, mDC were usually defined as Lin(CD3/CD19/CD14/CD56)⁻HLA-DR⁺CD11c⁺. This includes both BDCA-1⁺ and BDCA-3⁺ subsets. Our 11-color flow cytometric strategy made it possible to precisely define mDC subsets by avoiding contamination or exclusion of cells of interest. Indeed, we observed that both subsets expressed lineage markers, BDCA-1⁺ mDC expressing CD14 and subsets of the two mDC subpopulations expressing CD56, particularly BDCA-3⁺ mDC (Data not shown). Thus, we observed lower counts of circulating BDCA-1⁺ and even more significantly, of BDCA-3⁺ mDC counts in HIV-infected, untreated patients with viremia than in controls. This has been reported once as data not shown⁵⁵. Moreover these counts were normal in HIV-infected patients with cART-controlled viremia, as already found for CD11c mDC⁵⁶. Longitudinal studies will be needed to really prove that cART can restore these counts. Both mDC populations were also in lower proportions in the spleens from HIV-infected patients studied here than in those from uninfected patients. As expected, pDC counts were low in the blood from HIV-infected, untreated patients with viremia^{56,57}. They were normal in the spleens studied here, which had rather low proviral loads, confirming our previous study where high spleen pDC density was observed only with high proviral loads⁵⁸.

In summary, during chronic HIV infection with viremia uncontrolled by cART, the two types of mDC are depleted in the blood and the spleen, and pDC are depleted only in the blood. Concomitantly, we evidence here for the first time that the TNFα-producing M-DC8⁺ monocytes are expanded in the blood and the spleens from these patients and may have a major role in the maintenance of chronic immune activation leading to AIDS through their major production of TNFα in response to LPS⁵. This makes HIV infection a particular case of inflammatory disease. In Crohn's disease, anti-TNFα antibodies are used successfully to ablate intestinal inflammation, and anti-IL-12p40 are currently under trial. Similar approaches might be useful against the intestinal inflammation which fuels chronic immune activation during HIV infection. However, these antibodies induce a systemic immune suppression, which leads to susceptibility to mycobacteria, a side effect which may be dangerous during HIV infection. Rather than a global cytokine inhibition, targeting the cells that entertain a vicious immune activation cycle during HIV infection would be more specific. Therefore, our findings open the way to new therapeutic avenues using anti-M-DC8 monoclonal antibodies, which by specifically depleting M-DC8⁺ monocyte/macrophages, could resolve this chronic immune activation. This treatment would help patients under cART to reach a non-activated status similar to that of long-term non progressor or elite patients, who control HIV replication without anti-retroviral treatment.

Higher numbers of M-DC8+ monocytes were found in patients with HIV viremia compared to patients without by two converging methods: flow cytometry and in situ labeling. M-DC8+ monocytes were already found in inflamed gut mucosal tissues from patients with evolutive Crohn's disease³¹, in skin lesions from patients with psoriasis³² and in synovial lesions from patients with rheumatoid arthritis⁶¹. In HIV-infected, untreated patients, they were abnormally present within the marginal zone, i.e. in the lymphoid part of the spleen, where high viral replication takes place⁶². This indicates that they are driven to the lesions of this infection like to those of highly inflammatory diseases.

The present data show that M-DC8+ cells appear mostly responsible for the strong LPS-induced TNF-alpha overproduction in HIV-infected patients. Other data in the literature show that these cells appear mostly responsible for the overproduction of TNF-alpha in the lesions from Crohn's disease³¹, psoriasis³² and rheumatoid arthritis⁶¹. Therefore, the ground is laid to assume that depleting these cells indeed would be beneficial in these diseases where their strong TNF-alpha overproduction is related to pathogenesis.

TABLE 1
Blood and spleen samples, clinical data from patients

| | | | | | | | |
|---|---|---|---|---|---|---|---|
| Patient | HIV | | | | CD4 | log | |
| N^{o} | infection | Sexe | Age | Treatment (cART) | (cells/μl) | VL | Clinical data |

Blood samples:

| | | | | | | | |
|---|---|---|---|---|---|---|---|
| 1 | Yes | M | 42 | cART | ND | 1.00 | IKU |
| 2 | Yes | M | 37 | cART | 517 | 1.00 | JPE |
| 3 | Yes | M | 20 | cART | 523 | 1.00 | AKE |
| 4 | Yes | M | 47 | cART | 526 | 1.00 | BSK |
| 5 | Yes | M | 50 | cART | 548 | 1.00 | OOF |
| 7 | Yes | M | 50 | cART | 668 | 1.00 | GGE |
| 6 | Yes | M | 35 | cART | 693 | 1.00 | CKQ |
| 8 | Yes | M | 43 | cART | 793 | 1.00 | HEQ |
| 9 | Yes | M | 46 | / | 521 | 1.63 | 08GO(preVac) |
| 10 | Yes | F | 32 | / | 630 | 2.98 | 07BG(preVac) |
| 11 | Yes | F | 28 | / | 371 | 3.67 | 11LL(preVac) |
| 12 | Yes | F | 32 | / | 279 | 3.79 | 01AJ(preVac) |
| 13 | Yes | M | 49 | / | 596 | 4.17 | 15DD(preVac) |
| 14 | Yes | F | 38 | / | 544 | 4.2 | 05DO(preVac) |
| 16 | Yes | M | 40 | / | 779 | 4.25 | 03GE(preVac) |
| 15 | Yes | F | 54 | / | 311 | 4.25 | 02DS(preVac) |
| 17 | Yes | M | 39 | / | 300 | 4.27 | LME |
| 18 | Yes | M | 39 | / | 583 | 4.48 | MKS |
| 19 | Yes | M | 47 | / | 449 | 4.53 | 09BO(preVac) |
| 20 | Yes | F | 64 | / | 478 | 4.56 | 13DM(preVac) |
| 21 | Yes | F | 32 | / | 673 | 4.58 | 14TM(preVac) |
| 22 | Yes | M | 33 | / | 803 | 4.6 | 16DSTM(preVac) |
| 23 | Yes | M | 39 | / | 569 | 4.98 | HQO |

Spleen samples:

| | | | | | | | |
|---|---|---|---|---|---|---|---|
| DH33 | Yes | M | 63 | cART, VP16 | 294 | <50 | Castleman syndrome, Kaposi |
| | | | | | | | sarcoma, lipodystrophy |
| O | Yes | M | 36 | cART | 400 | <50 | ITP |
| N | Yes | M | 42 | cART, Foscarnet, | 13 | 18000 | ITP, hemophagocytosis, CMV |
| | | | | Rituximab, | | | infection, former cryptococcosis, |
| | | | | Corticoids, IvIg | | | mycosis |
| Q | Yes | M | ? | / | 110 | ND | ITP, hemophagocytosis, HBV |
| | | | | | | | hepatitis, salmonellosis, fever, |
| | | | | | | | asthenia, anorexia, weight loss |
| R | Yes | M | ? | / | 94 | ND | ITP |
| S | Yes | F | 69 | AZT | 312 | ND | ITP, pre-Castelman syndrome |
| X | No | F | ? | / | / | / | Nodules, angioma |
| A | No | F | 38 | Corticoids | / | / | ITP |
| C | No | M | 60 | /s | / | / | Pancreatic adenocarcinoma |
| E | No | F | 75 | Immunoglobulins | / | / | Evans syndrome (ITP + hemolytic |
| | | | | | | | anemia), toxic hepatitis |

cART = Combined Antiretroviral Treatment
ND: not done

### REFERENCES

Throughout this application, various references describe the state of the art to which this invention pertains. The disclosures of these references are hereby incorporated by reference into the present disclosure.
1. Veazey, R. S., et al. Gastrointestinal tract as a major site of CD4+ T cell depletion and viral replication in SIV infection. Science 280, 427-431 (1998).
2. Brenchley, J. M., et al. CD4+ T cell depletion during all stages of HIV disease occurs predominantly in the gastrointestinal tract. J Exp Med 200, 749-759 (2004).
3. Appay, V. & Sauce, D. Immune activation and inflammation in HIV-1 infection: causes and consequences. J Pathol 214, 231-241 (2008).
4. Boasso, A. & Shearer, G. M. Chronic innate immune activation as a cause of HIV-1 immunopathogenesis. Clin Immunol 126, 235-242 (2008).
5. Brenchley, J. M., et al. Microbial translocation is a cause of systemic immune activation in chronic HIV infection. Nat Med 12, 1365-1371 (2006).
6. Graziosi, C., et al. Kinetics of cytokine expression during primary human immunodeficiency virus type 1 infection. Proc Natl Acad Sci USA 93, 4386-4391 (1996).
7. Aukrust, P., et al. Tumor necrosis factor (TNF) system levels in human immunodeficiency virus-infected patients during highly active antiretroviral therapy: persistent TNF activation is associated with virologic and immunologic treatment failure. J Infect Dis 179, 74-82 (1999).
8. Aukrust, P., et al. Serum levels of tumor necrosis factor-alpha (TNF alpha) and soluble TNF receptors in human immunodeficiency virus type 1 infection—correlations to clinical, immunologic, and virologic parameters. J Infect Dis 169, 420-424 (1994).
9. Moir, S., Chun, T. W. & Fauci, A. S. Pathogenic mechanisms of HIV disease Annu Rev Pathol 6, 223-248 (2011).
10. Wang, F., et al. Interferon-gamma and tumor necrosis factor-alpha synergize to induce intestinal epithelial barrier dysfunction by up-regulating myosin light chain kinase expression. Am J Pathol 166, 409-419 (2005).
11. Ma, T. Y., et al. TNF-alpha-induced increase in intestinal epithelial tight junction permeability requires NF-kappa B activation. Am J Physiol Gastrointest Liver Physiol 286, G367-376 (2004).
12. Breen, E. C. Pro- and anti-inflammatory cytokines in human immunodeficiency virus infection and acquired immunodeficiency syndrome. Pharmacol Ther 95, 295-304 (2002).
13. Sanders, D. S. Mucosal integrity and barrier function in the pathogenesis of early lesions in Crohn's disease. J Clin Pathol 58, 568-572 (2005).
14. Griffin, G. E., Leung, K., Folks, T. M., Kunkel, S. & Nabel, G. J. Activation of HIV gene expression during monocyte differentiation by induction of NF-kappa B. Nature 339, 70-73 (1989).
15. Duh, E. J., Maury, W. J., Folks, T. M., Fauci, A. S. & Rabson, A. B. Tumor necrosis factor alpha activates human immunodeficiency virus type 1 through induction of nuclear factor binding to the NF-kappa B sites in the long terminal repeat. Proc Natl Acad Sci USA 86, 5974-5978 (1989).
16. Folks, T. M., Justement, J., Kinter, A., Dinarello, C. A. & Fauci, A. S. Cytokine-induced expression of HIV-1 in a chronically infected promonocyte cell line. Science 238, 800-802 (1987).
17. Pomerantz, R. J., Feinberg, M. B., Trono, D. & Baltimore, D. Lipopolysaccharide is a potent monocyte/macrophage-specific stimulator of human immunodeficiency virus type 1 expression. J Exp Med 172, 253-261 (1990).
18. Grassi, F., et al. Depletion in blood CD11c-positive dendritic cells from HIV-infected patients. AIDS 13, 759-766 (1999).
19. Dillon, S. M., et al. Blood myeloid dendritic cells from HIV-1-infected individuals display a proapoptotic profile characterized by decreased Bcl-2 levels and by caspase-3+ frequencies that are associated with levels of plasma viremia and T cell activation in an exploratory study. J Viol 85, 397-409 (2011).
20. Soumelis, V., et al. Depletion of circulating natural type 1 interferon-producing cells in HIV-infected AIDS patients. Blood 98, 906-912 (2001).
21. Crozat, K., et al. The XC chemokine receptor 1 is a conserved selective marker of mammalian cells homologous to mouse CD8alpha+ dendritic cells. J Exp Med 207, 1283-1292 (2010).
22. Bachem, A., et al. Superior antigen cross-presentation and XCR1 expression define human CD11c+CD141+ cells as homologues of mouse CD8+ dendritic cells. J Exp Med 207, 1273-1281 (2010).
23. Jongbloed, S. L., et al. Human CD141+ (BDCA-3)+ dendritic cells (DCs) represent a unique myeloid DC subset that cross-presents necrotic cell antigens. J Exp Med 207, 1247-1260 (2010).
24. Poulin, L. F., et al. Characterization of human DNGR-1+ BDCA3+ leukocytes as putative equivalents of mouse CD8alpha+ dendritic cells. J Exp Med 207, 1261-1271 (2010).
25. Thieblemont, N., Weiss, L., Sadeghi, H. M., Estcourt, C. & Haeffher-Cavaillon, N. CD14lowCD16high: a cytokine-producing monocyte subset which expands during human immunodeficiency virus infection. Eur J Immuno125, 3418-3424 (1995).
26. Ancuta, P., Weiss, L. & Haeflher-Cavaillon, N. CD14+CD16++ cells derived in vitro from peripheral blood monocytes exhibit phenotypic and functional dendritic cell-like characteristics. Eur J Immuno130, 1872-1883 (2000).
27. Ziegler-Heitbrock, L., et al. Nomenclature of monocytes and dendritic cells in blood. Blood 116, e74-80 (2010).
28. Auffray, C., Sieweke, M. H. & Geissmann, F. Blood monocytes: development, heterogeneity, and relationship with dendritic cells Annu Rev Immunol 27, 669-692 (2009).
29. Schakel, K., et al. A novel dendritic cell population in human blood: one-step immunomagnetic isolation by a specific mAb (M-DC8) and in vitro priming of cytotoxic T lymphocytes. Eur J Immuno128, 4084-4093 (1998).
30. Schakel, K., et al. 6-Sulfo LacNAc, a novel carbohydrate modification of PSGL-1, defines an inflammatory type of human dendritic cells. Immunity 17, 289-301 (2002).
31. de Baey, A., et al. A subset of human dendritic cells in the T cell area of mucosa-associated lymphoid tissue with a high potential to produce TNF-alpha. J Immunol 170, 5089-5094 (2003).
32. Hansel, A., et al. Human slan (6-sulfo LacNAc) dendritic cells are inflammatory dermal dendritic cells in psoriasis and drive strong TH17/TH1 T-cell responses. J Allergy Clin Immunol 127, 787-794 e781-789 (2011).
33. Sallusto, F. & Lanzavecchia, A. Efficient presentation of soluble antigen by cultured human dendritic cells is maintained by granulocyte/macrophage colony-stimulating factor plus interleukin 4 and downregulated by tumor necrosis factor alpha. J Exp Med 179, 1109-1118 (1994).
34. Romani, N., et al. Proliferating dendritic cell progenitors in human blood. J Exp Med 180, 83-93 (1994).
35. Buckner, C. M., Calderon, T. M., Willams, D. W., Belbin, T. J. & Berman, J. W. Characterization of monocyte maturation/differentiation that facilitates their transmigration across the blood-brain barrier and infection by HIV: implications for NeuroAIDS. Cell Immunol 267, 109-123 (2011).
36. Ancuta, P., et al. Microbial translocation is associated with increased monocyte activation and dementia in AIDS patients. PLoS One 3, e2516 (2008).
37. Ancuta, P., Moses, A. & Gabuzda, D. Transendothelial migration of CD16+ monocytes in response to fractalkine under constitutive and inflammatory conditions. Immunobiology 209, 11-20 (2004).
38. Pulliam, L., Gascon, R., Stubblebine, M., McGuire, D. & McGrath, M. S. Unique monocyte subset in patients with AIDS dementia. Lancet 349, 692-695 (1997).
39. Ancuta, P., et al. Fractalkine preferentially mediates arrest and migration of CD16+ monocytes. J Exp Med 197, 1701-1707 (2003).
40. Cotter, R., et al. Fractalkine (CX3CL1) and brain inflammation: Implications for HIV-1-associated dementia. J Neurovirol 8, 585-598 (2002).
41. Pereira, C. F., Middel, J., Jansen, G., Verhoef, J. & Nottet, H. S. Enhanced expression of fractalkine in HIV-1 associated dementia. J Neuroimmunol 115, 168-175 (2001).
42. Saha, R. N. & Pahan, K. Tumor necrosis factor-alpha at the crossroads of neuronal life and death during HIV-associated dementia. J Neurochem 86, 1057-1071 (2003).
43. de Baey, A., Mende, I., Riethmueller, G. & Baeuerle, P. A. Phenotype and function of human dendritic cells derived from M-DC8(+) monocytes. Eur J Immunol 31, 1646-1655 (2001).
44. Cassol, E., Cassetta, L., Alfano, M. & Poli, G. Macrophage polarization and HIV-1 infection. J Leukoc Biol 87, 599-608 (2010).
45. Shannon, M. F., Coles, L. S., Vadas, M. A. & Cockerill, P. N. Signals for activation of the GM-CSF promoter and enhancer in T cells. Crit. Rev Immunol 17, 301-323 (1997).
46. Trofimov, S., Pantsulaia, I., Kobyliansky, E. & Livshits, G. Circulating levels of receptor activator of nuclear factor-kappaB ligand/osteoprotegerin/macrophage-colony stimulating factor in a presumably healthy human population. Eur J Endocrinol 150, 305-311 (2004).
47. Gruber, M. F. & Gerrard, T. L. Production of macrophage colony-stimulating factor (M-CSF) by human monocytes is differentially regulated by GM-CSF, TNF alpha, and IFN-gamma. Cell Immunol 142, 361-369 (1992).
48. Cozzi-Lepri, A., et al. Resumption of HIV replication is associated with monocyte/macrophage derived cytokine and chemokine changes: results from a large international clinical trial. AIDS 25, 1207-1217 (2011).
49. von Sydow, M., Sonnerborg, A., Gaines, H. & Strannegard, O. Interferon-alpha and tumor necrosis factor-alpha in serum of patients in various stages of HIV-1 infection. AIDS Res Hum Retroviruses 7, 375-380 (1991).
50. Grimaldi, L. M., et al. Elevated alpha-tumor necrosis factor levels in spinal fluid from HIV-1-infected patients with central nervous system involvement. Ann Neurol 29, 21-25 (1991).
51. Fiala, M., et al. TNF-alpha opens a paracellular route for HIV-1 invasion across the blood-brain barrier. Mol Med 3, 553-564 (1997).
52. Aderka, D., Engelmann, H., Maor, Y., Brakebusch, C. & Wallach, D. Stabilization of the bioactivity of tumor necrosis factor by its soluble receptors. J Exp Med 175, 323-329 (1992).
53. Ryan, L. A., et al. Plasma levels of soluble CD14 and tumor necrosis factor-alpha type II receptor correlate with cognitive dysfunction during human immunodeficiency virus type 1 infection. J Infect Dis 184, 699-706 (2001).
54. Günther, C., et al. Caspase-8 regulates TNF-α-induced epithelial necroptosis and terminal ileitis. Nature 477, 335-339 (2011).
55. Chehimi, J., et al. Persistent decreases in blood plasmacytoid dendritic cell number and function despite effective highly active antiretroviral therapy and increased blood myeloid dendritic cells in HIV-infected individuals. J Immunol 168, 4796-4801 (2002).
56. Finke, J. S., Shodell, M., Shah, K., Siegal, F. P. & Steinman, R. M. Dendritic cell numbers in the blood of HIV-1 infected patients before and after changes in antiretroviral therapy. J Clin Immunol 24, 647-652 (2004).
57. Kamga, I., et al. Type I interferon production is profoundly and transiently impaired in primary HIV-1 infection. J Infect Dis 192, 303-310 (2005).
58. Nascimbeni, M., et al. Plasmacytoid dendritic cells accumulate in spleens from chronically HIV-infected patients but barely participate in interferon-alpha expression. Blood 113, 6112-6119 (2009).
59. McIlroy, D., et al. Investigation of human spleen dendritic cell phenotype and distribution reveals evidence of in vivo activation in a subset of organ donors. Blood 97, 3470-3477 (2001).
60. McIlroy, D., et al. Infection frequency of dendritic cells and CD4+ T lymphocytes in spleens of human immunodeficiency virus-positive patients. J Virol 69, 4737-4745 (1995).
61. Schakel K, von Kietzell M, Hansel A, et al. Human 6-sulfo LacNAc-expressing dendritic cells are principal producers of early interleukin-12 and are controlled by erythrocytes. Immunity. 2006; 24:767-777.
62. Hosmalin A, Samri A, Dumaurier M J, et al. HIV-specific effector CTL and HIV-producing cells co-localize in white pulps and germinal centers from infected patients. Blood. 2001; 97:2695-2701.

1. A M-DC8+ monocyte depleting agent for use in the prevention or treatment of a condition associated with a chronic hyperactivation of the immune system.

2. The M-DC8+ monocyte depleting agent for use according to claim 1, wherein said MDC8+ monocyte depleting agent is a M-DC8+ monocyte depleting antibody.

3. The M-DC8+ depleting agent for use according to claim 1, wherein said M-DC8+ monocyte depleting agent is an anti-M-DC8 antibody.

4. The M-DC8+ monocyte depleting agent for use according to claim 1, wherein said M-DC8+ monocyte depleting agent is an agent reducing or inhibiting the generation of MDC8+ monocytes from CD 14++CD 16− classical monocytes.

5. The M-DC8+ monocyte depleting agent for use according to claim 4, wherein said agent reducing or inhibiting the generation of MDC8+ monocytes from CD14++CD16− classical monocytes is an antagonist of the GM-CSF receptor (GMCSFR) or the M-CSF receptor (M-CSFR) or a combination thereof.

6. The M-DC8+ depleting agent for use according to claim 1, wherein said condition associated with a chronic hyperactivation of the immune system is a condition mediated by a TNFa overproduction selected from the group consisting of a chronic inflammatory or infectious disease.

7. The M-DC8+ depleting agent for use according to claim 6, wherein said a chronic inflammatory disease is selected from the group consisting of rheumatoid arthritis, psoriasis, psoriatic arthritis, ankylosing spondylitis and inflammatory bowel disease (IBD) including ulcerative colitis, Crohn's disease and metabolic syndromes including atherosclerosis, obesity, diabetes and hypertension.

8. The M-DC8+ depleting agent for use according to claim 6, wherein said a chronic infectious disease is selected from the group consisting of HIV infection and other chronic viral diseases such as CMV, EBV and other herpes virus infections, HTL V-1 and other retroviral infections, and mycobacterial infections.
